(19) United States
(12) Patent Application Publication
(10) Pub. No.: US 2016/0106342 A1
(43) Pub. Date: Apr. 21, 2016

(54) SYSTEMS AND METHODS FOR MONITORING LUNG FUNCTION

(71) Applicants: Mehdi RAZAVI, Houston, TX (US); Christopher Alexander AREVALOS, Houston, TX (US); William Thomas POST, Kalamazoo, MI (US); Keri PETERSEN, College Station, TX (US); Travis SCHAEFER, College Station, TX (US); Dan YANG, College Station, TX (US); Keifer GEERS, College Station, TX (US)

(72) Inventors: Mehdi RAZAVI, Houston, TX (US); Christopher Alexander AREVALOS, Houston, TX (US); William Thomas POST, Kalamazoo, MI (US); Keri PETERSEN, College Station, TX (US); Travis SCHAEFER, College Station, TX (US); Dan YANG, College Station, TX (US); Keifer GEERS, College Station, TX (US)

(21) Appl. No.: 14/918,215

(22) Filed: Oct. 20, 2015

Related U.S. Application Data
(60) Provisional application No. 62/066,060, filed on Oct. 20, 2014.

(51) Int. Cl. A61B 5/097; A61B 5/00

(52) U.S. Cl. CPC A61B 5/097; A61B 5/7271; A61B 5/702; A61B 5/6824; A61B 5/0004; A61B 5/0022; A61B 5/742

(57) ABSTRACT

A measurement assembly of a personal health monitoring system for measuring the lung capacity of a person. In an embodiment, the measurement assembly includes a sensor assembly includes a sensor that is configured to sense the force of a fluid exhaled by the person onto the sensor and output a force measurement. In addition, the measurement assembly includes a control assembly coupled to the force sensing assembly, the control assembly configured to receive the force measurement from the sensor. Further, the measurement assembly includes a housing configured to support each of the sensor assembly and the control assembly. The sensor is disposed on an external surface of the housing.

### CROSS-REFERENCE TO RELATED APPLICATIONS

This application claims benefit of U.S. provisional patent application Ser. No. 62/066,060 filed Oct. 20, 2014 and entitled “Force Sensing Peak Flow Meter,” which is hereby incorporated herein by reference in its entirety for all purposes.

### STATEMENT REGARDING FEDERALLY SPONSORED RESEARCH OR DEVELOPMENT

Not applicable.

### BACKGROUND

This disclosure generally relates to personal health monitoring. More particularly, this disclosure relates to systems and methods for monitoring the lung function of a person.

Individuals who suffer from reduced lung function due to, for example, chronic obstructed pulmonary disease (COPD), asthma, etc. typically must monitor the performance of their lungs over time to provide the treating physician with vital information regarding the progression of the patient's condition. In addition, individuals engaged in physical activity (e.g., athletes) often desire to monitor their lung function and any changes thereto based on their physical activity they are engaged in, to track physical and/or athletic performance. Conventionally, such individuals track lung function through utilizing peak sensing flow meters that measure the volume of exhaled air over a defined period of time and then relate this volume to a flow rate.

### BRIEF SUMMARY OF THE DISCLOSURE

Some embodiments are directed a measurement assembly for measuring the lung capacity of a person to. In an embodiment, the measurement assembly includes a sensor assembly including a sensor that is configured to sense the force of a fluid exhaled by the person onto the sensor and output a force measurement. In addition, the measurement assembly includes a control assembly coupled to the force sensing assembly, the control assembly configured to receive the force measurement from the sensor. Further, the measurement assembly includes a housing configured to support each of the sensor assembly and the control assembly. The sensor is disposed on an external surface of the housing.

Other embodiments are directed to a measurement assembly for measuring the lung capacity of a person. In an embodiment the measurement assembly includes a sensor assembly including a sensor that is configured to sense the force of a fluid exhaled by the person onto the sensor and output a force measurement. In addition, the measurement assembly includes a control assembly electrically coupled to the force sensing assembly, the control assembly configured to receive the force measurement from the sensor. Further, the measurement assembly includes a housing configured to support each of the sensor assembly and the control assembly. The housing includes a receptacle configured to receive and house a smartphone therein.

Still other embodiments are directed to a personal health monitoring system for monitoring the lung capacity of a person. In an embodiment, the system includes a sensor assembly including a sensor that is configured to sense the force of a fluid exhaled by the person onto the sensor and output a force measurement. In addition, the system includes a housing configured to support the sensor assembly and the control assembly. The sensor is disposed on an external surface of the housing. Further, the system includes a computing device coupled to the sensor assembly. The computing device includes a display that is configured to display information indicative of the force measurement.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a front, schematic view of a personal health monitoring system in accordance with at least some embodiments;

FIG. 2 is a side, schematic view of the personal health monitoring system of FIG. 1;

FIG. 3 is a rear, schematic view of the personal health monitoring system of FIG. 1;

FIG. 4 is a block diagram of the personal health monitoring system of FIG. 1;

FIG. 5 is a block diagram of a method for monitoring the lung function of a person in accordance with at least some embodiments;

FIG. 6 is a front, schematic view of another personal health monitoring system in accordance with at least some embodiments; and

FIG. 7 is a front, schematic view of still another personal health monitoring system in accordance with at least some embodiments.

### DETAILED DESCRIPTION OF EXEMPLARY EMBODIMENTS

The following discussion is directed to various exemplary embodiments. However, one of ordinary skill in the art will understand that the examples disclosed herein have broad application, and that the discussion of any embodiment is meant only to be exemplary of that embodiment, and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, as used herein, the terms “axial” and “axially” generally mean along or parallel to a given axis (e.g., central axis of a body or a port), while the terms “radial” and “radially” generally mean perpendicular to the given axis. For instance, an axial distance refers to a distance measured along or parallel to the axis, and a radial distance means a distance measured perpendicular to the axis.

As previously described, individuals who wish to monitor their lung function (e.g., in order to monitor the progression of a disease such as asthma or COPD, to track athletic performance, etc.) typically utilize a peak sensing flow meter that measures the volume of air that is exhaled by the patient to determine the flow rate that is output by the lungs of the person. However, such flow sensing devices are relatively large, and therefore are less convenient to use. As a result, many individuals utilizing these sorts of devices are less likely to regularly and consistently take measurements of their lung function, such that the patient and/or the treating physician are given an incomplete (and therefore possibly insufficient) view of the patient's lung performance over time. Therefore, embodiments disclosed herein employ personal health monitoring systems that utilize force sensors to measure the force of air exhaled by the user, which may then be related to the flow rate of exhaled air. Because the force sensing components are much more compact than the volume measuring components typically utilized in conventional peak flow meters, the personal health monitoring systems disclosed herein may be smaller in size and thus more convenient to use then these conventional systems.

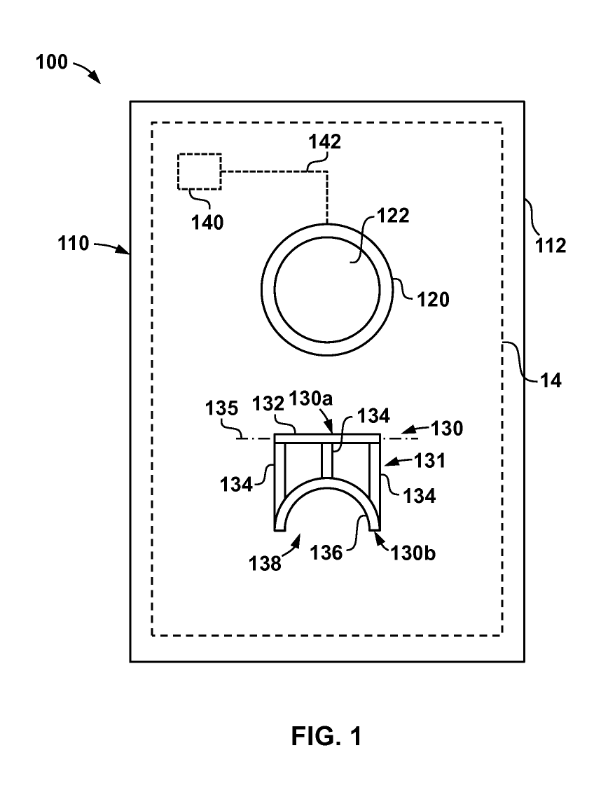

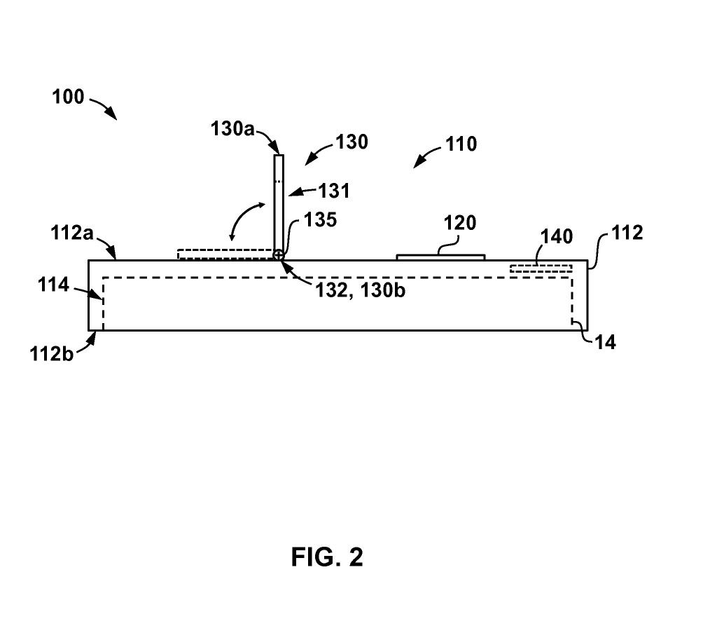

Referring now to FIGS. 1-3, an embodiment of a personal health-monitoring system 100 for monitoring the lung function of a person in accordance with at least some embodiments is schematically shown. System 100 generally includes a computing device 14 and a measurement assembly 110 coupled to device 14. In this embodiment, device 14 comprises a smartphone; however, in other embodiments, device 14 may comprise any suitable computing device including, for example, a tablet computer, a laptop, a separate medical device, or some combination thereof. Measurement assembly 110 generally includes, a housing 112, a sensor assembly 120, a user interaction assembly 130, and a control assembly 140 (note: control assembly 140 is schematically shown with a hidden line in both FIGS. 1 and 2).

Housing 112 may comprise any suitable housing or member for supporting and holding the assemblies 120, 130, and/or 140 while still complying with the principles disclosed herein. In this this embodiment, housing 112 comprises a protective case for computing device 14 (note: device 14 is shown with a hidden line in both FIGS. 1 and 2). Thus, in this embodiment housing 112 comprises a protective case for a smartphone. However, in other embodiments, housing 112 may be a protective case for another type of computing device (e.g., tablet computer, laptop, etc.). As a result, housing 112 may be constructed out of any material(s) that is suitable for protective cases for electronic equipment, such as, for example, polymers, rubbers (natural, synthetic, etc.), etc. In this embodiment, housing 112 comprises a first or closed side 112a, a second or open side 112b opposite closed side 112a, and a receptacle 114 extending into housing 112 from open side 112a.

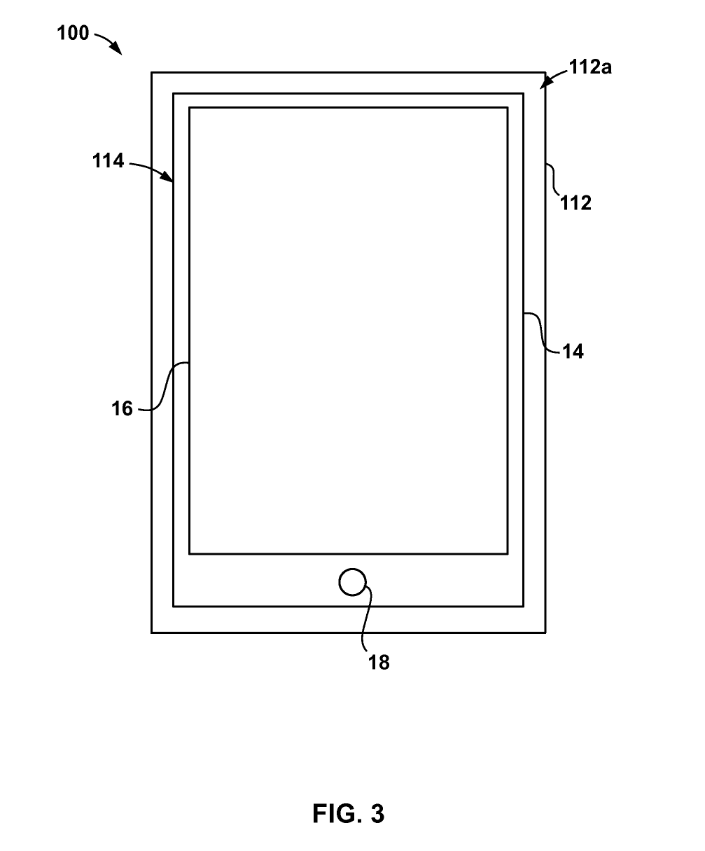

During operations, computing device 14 is inserted within receptacle 114 from open side 112a and secured therein through any suitable connection, such as, for example, an interference fit, snaps, adhesive, etc. As is best shown in FIG. 3, computing device 14 includes a display 16 (which may, in some embodiments, comprise a touch sensitive display) that is visible and accessible by a user through the open end 112b of housing 112. In addition, computing device 14 may also include one or more buttons or other user interface features 18 that are visible and accessible through open end 112b of housing 112.

Referring now to FIGS. 1 and 2, sensor assembly 120 is disposed on closed side 112a of housing 112 and includes a sensor 122 that is configured to sense the force or pressure of air exhaled (e.g., blown) by a user (e.g., a patient) toward sensor 120 during operation. Sensor 122 may be any suitable type of sensor for measuring the force or pressure of air expired and/or exhaled by a user. For example, in some embodiments sensor 122 may include a membrane, string, one or more piezoelectric elements, one or more piezoresistive elements, one or more force sensing resistors, one or more pressure sensors, one or more force sensors, and one or more strain gauges, or combinations thereof. Thus, as shown herein, sensor 122 is disposed along an external surface of housing 112 (e.g., closed side 112b) so that a user may easily exhale directly onto sensor 122 during operations.

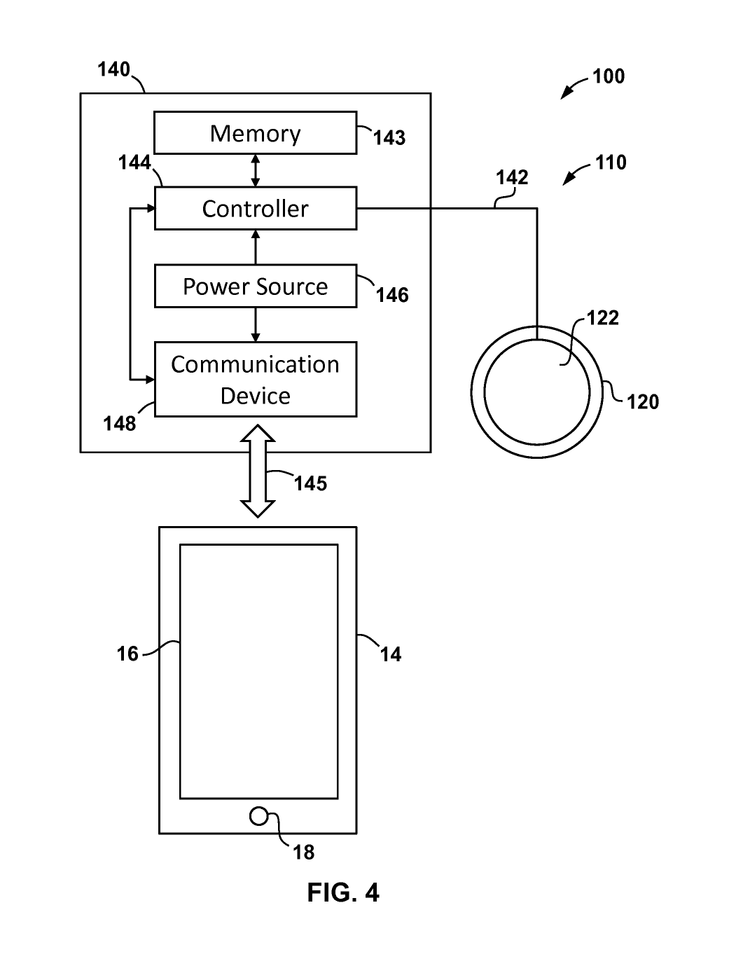

Referring now to FIGS. 1 and 4, control assembly 140 may comprise any suitable device or assembly which is capable of receiving an electrical or mechanical signal and transmitting the received signal to another device (e.g., computing device 14). In some embodiments, control assembly 140 is configured to receive a mechanical signal (e.g., deflection of the membrane or string in sensor assembly 120) and convert this mechanical signal into an electrical signal. In other embodiments, control assembly 140 is configured to receive an electrical signal (e.g., one that is already converted from a mechanical signal by the sensor assembly 120). In particular, as shown in FIG. 4, in this embodiment, control assembly 140 includes a controller 144, a memory 143, a power source 146, and a communication device 148.

The controller 144 executes software provided on memory 143, and upon executing the software on memory 143 provides the control assembly 140 with all of the functionality described herein. The memory 143 may comprise volatile storage (e.g., random access memory), non-volatile storage (e.g., flash storage, read only memory, etc.), or combinations of both volatile and non-volatile storage. Data consumed or produced by the software can also be stored on memory 143. For example, measured force values can be stored on memory 143 pending transmission (wireless or wired) to computing device 14. Controller 144 is coupled to sensor 122 with a conductor 142, which may comprise any suitable electrical conductor (e.g., a metal wire). In some embodiments, conductor 142 may be a fiber optic cable. In still other embodiments, controller 144 may communicate with sensor 122 via a wireless signal (e.g., one or more of the wireless signal types discussed herein).

The power source 146 may comprise a battery (disposable or rechargeable), a charged capacitor, a wireless power receiver (e.g., inductive coil, etc.), or other sources of electrical power. The power source 146 provides electrical power to the other components within control assembly 140 (e.g., controller 144, communication device 148, etc.) and in some embodiments may provide power to one or more of the sensing elements within sensor assembly 120 (e.g., sensor 122).

The communication device 148 may be implemented in accordance with any suitable wireless protocol (e.g., BLUETOOTH®, WiFi, near field communications, radio-frequency communications, etc.) or wired communication system (e.g., an electrical conductor, fiber optic cable, etc.). In this embodiment, communication device 148 is a wireless communication device that is configured to communicate with computing device 14, through a wireless signal path 145. The communication device 148 may be capable of transmitting only, or may be capable of transmitting and receiving. The controller 144 causes the communication device 148 to transmit values of measured force from sensor 122 in sensor assembly 120 to computing device 14 via wireless signal path 145. The communication device 148 may be a bi-directional device to permit outgoing transmissions of data, as well as receive incoming commands from a computing device (e.g., computing device 14). For example, computing device 14 may send a command to the controller 144 via the communication device 148 to command controller 144 to receive output force measurements (e.g., electrical output signals) from sensor 122 or to transmit previously stored data (e.g., previously stored force measurements from sensor 122).

Referring still to FIGS. 1 and 4, during operations a user may operate software stored and executed on computing device 14 and initiate a peak force measurement operation. Alternatively, a user may directly interact with measurement assembly 110 to initiate a peak force measurement operation. Regardless, a user (e.g., a patient) may select to take measurement of a peak force generated by the user's lungs (e.g., by selecting a button or defined region of display 16). The computing device may then output a command to communication device 148 in control assembly 140 (e.g., via wireless communication path 145) to receive a force measurement from sensor 122. The command signal is routed to the controller 144, which then allows itself to receive an input signal from sensor 122 in sensing assembly 120. The user then blows or forcibly exhales onto sensor 122, which senses the force of the exhaled air via one or more of the sensing methods discussed above and routes a measurement signal through conductor 142 back to controller 144.

Controller 144 may then execute software that is stored on memory 143 to process the received signals. Processing of the received measurement signals from sensor 122 may include, for example, converting the received signals from an electrical signal into force measurements, calculating peak or maximum value, calculating an average force value, compared the measured values to a previously calculated base value (the calculation of which is discussed in more detail below), etc. In other embodiments, controller 144 may simply receive the signals from sensor 122 and command communication device 148 to output the received signals to computing device 14 where they are then processed. In still other embodiments, controller 144 may perform only some of the processing steps noted above and then command communication device 148 to output the received signals to computing device 14 for further processing and/or storage. In some embodiments, controller 144 may store some or all of the received and/or processed data from sensor 122 on memory 143.

Referring again to FIGS. 1 and 2, user interaction assembly 130 comprises a hinged member or kickstand 131 that is rotatably mounted to housing 112. In particular, member 131 includes a first or proximate end 130a pivotally coupled to closed side 112a of housing 112, and a second or distal end 130b opposite to proximate end 130a. Proximate end 130a comprises a hinge 132 that is configured to allow distal end 130b to rotate about an axis of rotation 135 relative to housing 112. Distal end 130b comprises a curved member 136 further defining a chin rest 138. As will be described in more detail below, chin rest 138 is configured to receive the chin of a patient in order to ensure proper and consistent spacing of a patient's mouth from sensor 122 in assembly 120 during operations. Chin rest 138 and hinge 132 are coupled to one another through a plurality of bridging members 134. In this embodiment, there are a total of three bridging members 134 extending substantially perpendicular to axis 135; however, it should be appreciated that in other embodiments, the number and orientation or bridging members 134 may be greatly varied.

As is best shown in FIG. 2, during operations, assembly 130 is rotatable between a first or stored position (shown with hidden line) in which the assembly 30 is substantially aligned or, in some embodiments, fully or partially withdrawn within the housing 112, and a second or deployed position where assembly 130 extends outward or away from housing 112. Further, it should be appreciated that in other embodiments, assembly 130 is not included with device 10 while still complying with the principles disclosed herein. Still further, in embodiments not including assembly 130, other techniques or devices may be used to ensure proper spacing between the patient's mouth and the sensor assembly 120. For example, in some embodiments, an instruction may be delivered to the patient to use a number of the patient's fingers or a portion of the patient's hand to provide the necessary spacing between the patient's mouth and the sensor assembly 120 during operation.

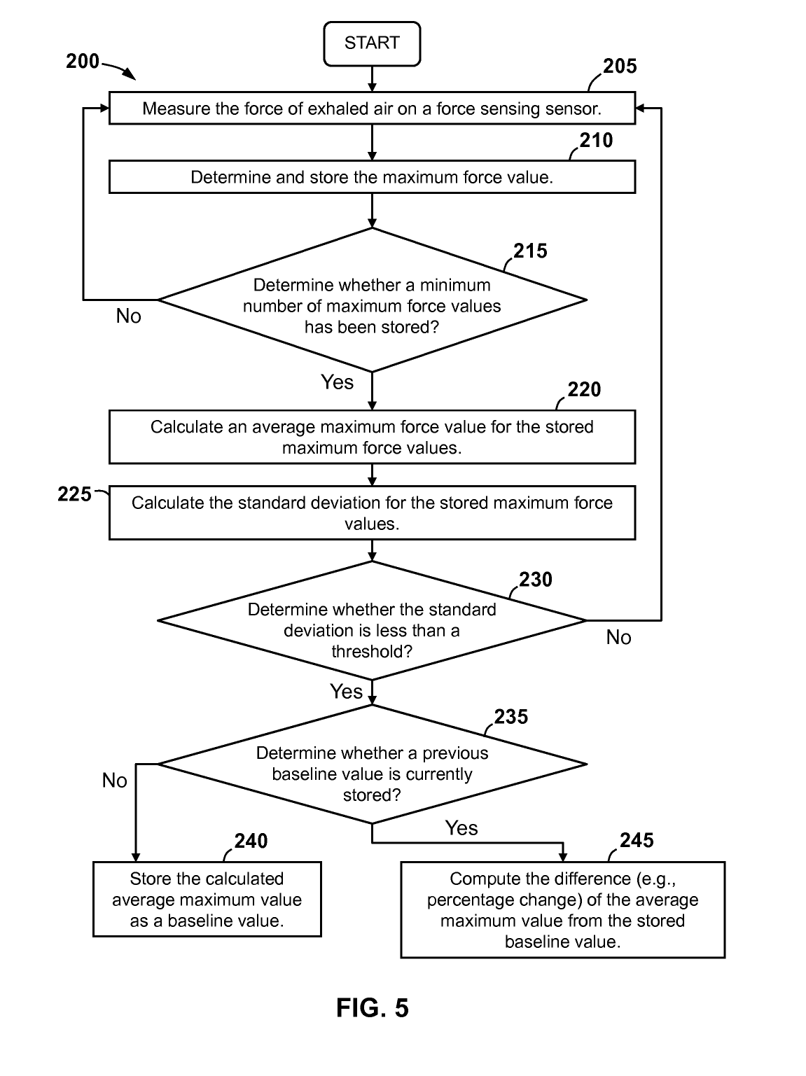

Referring now to FIG. 5, a method 200 for monitoring the lung function of an individual in accordance with at least some embodiments is shown. In the description below, method 200 will be described as being performed with personal health monitoring system 100 shown in FIGS. 1-4; however, it should be appreciated that method 200 may be performed with other personal health monitoring systems in other embodiments (e.g., such as systems 300, 400 discussed below).

Initially, at 205, the method 200 includes sensing the force of exhaled air from a user. The force of the exhaled air may be sensed by a sensor such as sensor 122 in sensor assembly 120, previously described. The force of the exhaled air may be measured for some predetermined period of time (e.g., 5-10 seconds) or may be measured for as long as the measured force (or pressure) is above some minimum threshold value (which may be set so as to distinguish purposely exhaled air from the mouth of the user from normal air flow within a given environment). Next, at 210, the maximum or peak force value that is measured during 205 is determined and stored (e.g., in memory 143 in control assembly 140 and/or in another suitable memory or storage device in computing device 14).

Thereafter, at 215, method 200 determines whether a minimum number of maximum force values have been stored. For example, in some embodiments, at least three readings are stored for calculating or establishing a baseline or comparing against a previously stored baseline; however, in other embodiments, the minimum number of stored values may be fewer or greater than three (e.g., 1, 2, 4, 5, etc.). If it is determined that the minimum number of values have not been stored (i.e., the determination at 215 is No), method 200 returns to steps 205 and 210, where another maximum force value is stored (i.e., the force is measured at 205, and the maximum force value from the measurement in 205 is determined and stored at 210).

If, on the other hand, the minimum number of values have been stored (i.e., the determination at 215 is Yes), method continues on to 220, where an average of the stored maximum force values is calculated. Next, at 225 the standard deviation of the stored maximum force values is calculated. Thereafter, at 230, it is determined whether the standard deviation calculated at 225 is less than a threshold. The threshold in 230 may be any suitable value for evaluating the quality of measurements obtained at 205. For example, in some embodiments, the threshold may be expressed as a percentage value of the average maximum value calculated at 220 (e.g., such as 10%).

If the standard deviation calculated at 225 is more than or equal to the threshold in 230 (i.e., if the determination in 230 is No), then method 200 returns to steps 205 to take additional measurements. If, on the other hand, the standard deviation calculated at 225 is less than the threshold 230 (i.e., if the determination in 230 is Yes), then method 200 advances to 235 where it is determined whether a previous baseline value has been recorded or stored (e.g., stored in memory 143 in control assembly 140 and/or in another suitable memory or storage device in computing device 14). If a previous baseline value has not already been stored (i.e., if the determination in 235 is No), then the average maximum force value calculated at 220 is stored as a baseline value. If, on the other hand, a previous baseline value has already been stored (i.e., if the determination in 235 is Yes), then a difference between the average maximum force value calculated at 220 and the baseline value is computed at 245. In some embodiments, the difference between the average maximum force value and the baseline value is computed as a percentage change.

In some embodiments, a user may operate system 100 to indicate a difference (e.g., percentage change) of the current average maximum force value (e.g., the value calculated at 220) and the last average maximum force value, rather than comparing the average maximum force value to the historical baseline value.

During use of the system 100 according to method 200, the user may see any displayed information (e.g., the maximum force values from 210, the average maximum force value from 220, the standard deviation from 225, the baseline value from 240, the difference value from 245, etc.) on display 16 of computing device 14. In some embodiments, during use of system 100 according to method 200, sensor assembly 120 may perform the measurements at 205 and control assembly 140 may perform all of the remaining calculations, and analysis described above (e.g., steps 210-245). In these embodiments, control assembly 140 (e.g., communication device 148) may simply communicate the resulting numerical values (e.g., the maximum force values from 210, the average maximum force value from 220, the standard deviation from 225, the baseline value from 240, the difference value from 245, etc.) to computing device 14 for further display to the user (e.g., on display 16). Alternatively, in other embodiments, sensor assembly 120 may perform the measurements at 205, the measurements may be communicated to computing device 14 via control assembly 140 (e.g., via communication device 148), and then computing device 14 may perform all of the remaining calculations and analysis (e.g., steps 210-245). In still other embodiments, sensor assembly 120 may perform the measurements at 205, and then control assembly 140 and computing device 14 may together perform the remaining calculations and analysis (e.g., steps 210-245)—with control assembly 140 performing some of the steps 210-245 and computing device 14 performing the remaining steps 210-245 that are not performed by control assembly 140.

Further, it should also be appreciated that method 200 may also include a step (or steps) for converting or relating the force measurements in 205, the maximum force values in 210, 220, 240, and/or the percentage change in 245 to flow rate values. This computation may be performed by control assembly 140 and/or computing device 14, and is based on known relationships and correlations. Thus, the details of this computation are not provided in detail herein in the interests of brevity. Certain parameters required for the conversion of measured force to flow rate (e.g., the surface area of the sensor, average density of air exhaled by a person, etc.) are stored or saved on memory 143 and/or computing device 14, or both. Thus, in some embodiments, numeral values displayed to the user (e.g., on display 16) (which may include the measurements in 205, the values in 210, 220, 240, and 245, etc.) may be expressed in terms of flow rate either in addition to or in lieu of force (or pressure).

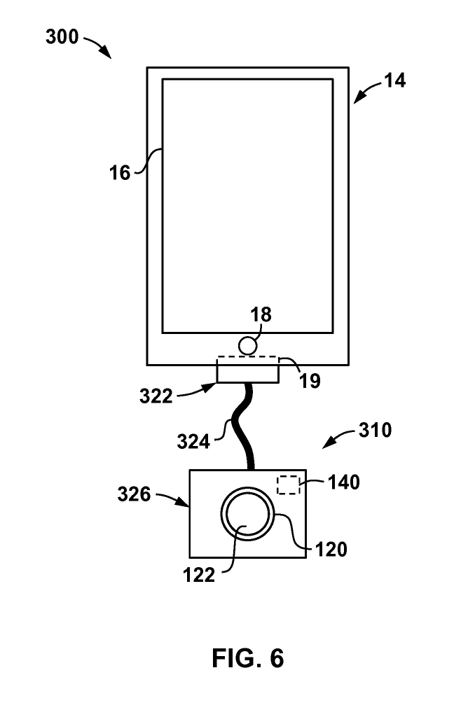

Referring now to FIG. 6, where another embodiment of a personal health-monitoring system 300 is schematically shown. In general, system 300 includes computing device 14 previously described, and a measurement assembly 310 coupled to computing device 14.

Measurement assembly 310 includes a housing 326 that supports sensor assembly 120, and also houses control assembly 140 (where sensor assembly 120 and control assembly 140 are each the same as previously described above). Housing 326 is a structural member that houses and protects assemblies 120, 140 (as well as supporting and other equipment and components). Thus, housing 326 may comprise a suitable material for protecting assemblies 120, 140 from damage, such as, for example, a polymer, metals, composite materials (e.g., carbon fiber), etc. As with system 100, sensor 122 in sensor assembly 120 is disposed on or along an external surface of housing 326 so as to allow a user to more easily exhale directly onto sensor 122 during operations.

Each of the sensor assembly 120 and control assembly 140 are coupled to computing device 14 through a conductor 324 that extends from housing 326 to a connector 322. Conductor 324 may be any suitable conductor configured to transmit or conduct an electrical signal (e.g., one or more electrically conductive wires). In addition, connector 322 may be any suitable electrical connector for electrically coupling one electrical device to another (e.g., a pinned connector, a universal serial bus (USB) connector, etc.). In this embodiment, connector 322 is inserted within a mating receptacle 19 on computing device 19 that is configured to receive and mate with connector 322 and includes one or more electrical connections that engage with the electrical connections on connector 322 to thereby electrically couple sensor assembly 120 and control assembly 140 to computing device 14.

Operations with system 300 are substantially the same as those described above for system 100 (e.g., see method 200 in FIG. 5 the associate text above). Therefore, a detailed description of the operations with system 300 is omitted in the interests of brevity. It should be appreciated that in some embodiments, conductor 324 may be omitted and the electronic components housed within and/or supported by housing 326 (e.g., sensor assembly 120, control assembly 140) are electrically coupled to computing device 14 through a wireless connection (e.g., wireless connection 145 discussed above).

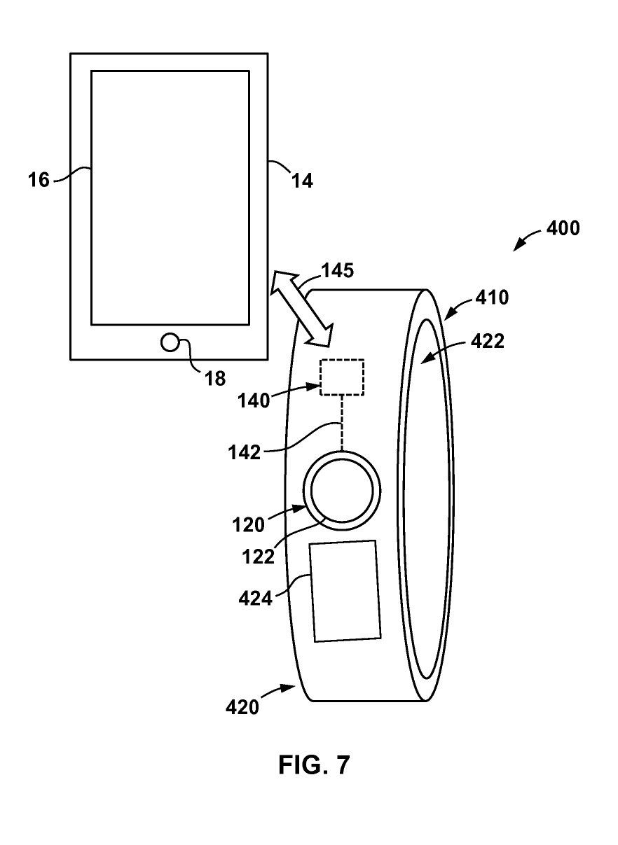

Referring now to FIG. 7, where another embodiment of a personal health-monitoring system 400 is schematically shown. In general, system 400 includes computing device 14 previously described, and a measurement assembly 410.

In this embodiment, measurement assembly 410 includes an annular ring-shaped housing member 420 that includes a through passage 422 extending therethrough. Housing member 420 may be worn on the wrist of a user (e.g., in the same or similar manner as a wrist watch), and houses and supports sensor assembly 120 and control assembly 140 (where sensor assembly 120 and control assembly 140 are each the same as previously described above). In addition, measurement assembly 410 also includes a display 424 that is disposed on (or carried by) housing member 420. Display 424 may be any type of display suitable for displaying images and information thereon (e.g., a liquid crystal display (LCD), a plasma display). In some embodiments, display 424 may be touch sensitive. Housing member 420 is a structural housing to house and protect assemblies 120, 140 and display 424 (as well as supporting and other equipment and components). Thus, housing member 420 may comprise a suitable material for protecting assemblies 120, 140 and dosplay 424 from damage, such as, for example, a polymer, metals, composite materials (e.g., carbon fiber), etc. As with system 100, sensor 122 in sensor assembly 120 is disposed on or along an external surface of housing 420 so as to allow a user to more easily exhale directly onto sensor 122 during operations.

Assemblies 120, 140 are electrically coupled to display 424 through internal conductors or a wireless connection (not specifically shown), and are electrically coupled to computing device 14 through wireless connection path 145, previously described above. In some embodiments, assemblies 120, 140 and/or display 424 may be electrically coupled to computing device 14 through a wired connection (e.g., through an electrical conductor similar to conductor 324 previously described above).

Operations with system 400 are substantially the same as those described above for system 100 (e.g., see method 200 in FIG. 5 the associate text above). Therefore, a detailed description of the operations with system 400 is omitted in the interests of brevity.

It should be further be appreciated that in some embodiments, a personal health monitoring system in accordance with the embodiments disclosed herein (e.g., systems 100, 300, 400) may include a “first use” procedure or application that is run by control assembly 140, computing device 14, or both which allows a particular user or patient to input some important personal information which may include, for example, age, height, sex, weight, etc. In some embodiments, the program displays a set of instructions for proper use of the system. These instructions may be textual, graphical, pictorial, or some combination thereof.

In the manner described, through use of a personal health monitoring system in accordance with the embodiments disclosed herein (e.g., systems 100, 300, 400), a person my monitor their lung function so as to, for example, track the progression of a disease (e.g., COPD, asthma, etc.) or track athletic performance (e.g., such as tracking changes in lung capacity). In addition, because of the relative compactness of the force sensing components (e.g., sensors 122 in sensor assembly 120) utilized in the personal health monitoring systems disclosed herein (e.g., systems 100, 300, 400), use and transportation of the presently disclosed personal health monitoring systems is more convenient that other conventional systems that utilize volume measurement components.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the invention. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A measurement assembly for measuring the lung capacity of a person, the measurement assembly comprising:
a sensor assembly including a sensor that is configured to sense the force of a fluid exhaled by the person onto the sensor and output a force measurement;
a control assembly coupled to the sensor assembly, the control assembly configured to receive the force measurement from the sensor; and
a housing configured to support each of the force sensing assembly and the control assembly, wherein the sensor is disposed on an external surface of the housing.

2. The measurement assembly of claim 1, wherein the control assembly includes a controller configured to:
receive the force measurement;
determine an average maximum force exhaled by the person; and
determine a difference between the average maximum force exhaled by the person and a baseline value.

3. The measurement assembly of claim 1, wherein the housing comprises a protective case for a computing device.

4. The measurement assembly of claim 3, wherein the housing comprises a receptacle configured to receive and house the computing device.

5. The measurement assembly of claim 1, further comprising a chin rest coupled to the housing, wherein the chin rest is configured to receive the chin of the person when the person is exhaling onto the sensor.

6. The measurement assembly of claim 5, wherein the chin rest is defined by a hinged member pivotally coupled to the housing;
wherein the hinged member is pivotable between a first position where the hinged member extends along the external surface of the housing and a second position, where the hinged member extends away from the external surface of the housing.

7. The measurement assembly of claim 1, further including an electrical connector configured to mate and engage with a receptacle in a computing device; wherein the electrical connector is electrically coupled to the sensor assembly and the control assembly through a conductor.

8. The measurement assembly of claim 1, wherein the housing comprises an annular member that is configured to be disposed about the wrist of the person.

9. The measurement assembly of claim 1, wherein the control assembly includes a communication device configured to communicate with a computing device.

10. The measurement assembly of claim 1, wherein the communication device is configured to communicate with the computing device through a wireless signal.

11. A measurement assembly for measuring the lung capacity of a person, the measurement assembly comprising:
a sensor assembly including a sensor that is configured to sense the force of a fluid exhaled by the person onto the sensor and output a force measurement;
a control assembly electrically coupled to the sensor assembly, the control assembly configured to receive the force measurement from the sensor; and
a housing configured to support each of the force sensing assembly and the control assembly, wherein the housing includes a receptacle configured to receive and house a smartphone therein.

12. The measurement assembly of claim 11, wherein the control assembly includes a controller configured to:
receive the force measurement;
determine an average maximum force exhaled by the person; and
determine a difference between the average maximum force exhaled by the person and a baseline value.

13. The measurement assembly of claim 11, wherein the control assembly includes a communication device configured to communicate with the smartphone.

14. The measurement assembly of claim 13, wherein the communication device is configured to communicate with the smartphone through a wireless signal.

15. The measurement assembly of claim 11, further comprising a chin rest coupled to the housing, wherein the chin rest is configured to receive the chin of the person when the person is exhaling onto the sensor.

16. A personal health monitoring system for monitoring the lung capacity of a person, the system including:
a sensor assembly including a sensor that is configured to sense the force of a fluid exhaled by the person onto the sensor and output a force measurement;
a housing configured to support the sensor assembly and the control assembly, wherein the sensor is disposed on an external surface of the housing;
a computing device coupled to the sensor assembly, wherein the computing device includes a display that is configured to display information indicative of the force measurement.

17. The system of claim 16, wherein the computing device comprises one of a smartphone and a tablet computer;
wherein the housing includes a closed side, an open side opposite the closed side, and a receptacle extending into the housing from the open side;
wherein the computing device is received within the receptacle such that the display is accessible through the open side;
wherein the external surface of the housing is on the closed side.

18. The system of claim 16, wherein the computing device comprises one of a smartphone and a tablet computer.

19. The system of claim 16, wherein the housing comprises an annular member that is configured to be disposed about the wrist of the person.

20. The system of claim 16, further comprising a control assembly, including:
a controller configured to receive the force measurement from the sensor;
a communication device coupled to the controller and configured to communicate with the computing device.
